(19) United States
(12) Patent Application Publication
(10) Pub. No.: US 2009/0169471 A1
(43) Pub. Date: Jul. 2, 2009

(54) PARTICLES FOR INJECTION AND PROCESSES FOR FORMING THE SAME

(75) Inventors: Robert E. Richard, Wrentham, MA (US); John E. O'Gara, Ashland, MA (US); Sonali Puri, Ashland, MA (US)

Correspondence Address: MAYER & WILLIAMS PC, 251 NORTH AVENUE WEST, 2ND FLOOR, WESTFIELD, NJ, 07090, US

(73) Assignee: BOSTON SCIENTIFIC SCIMED, INC., Maple Grove, MN (US)

(21) Appl. No.: 12/339,588

(22) Filed: Dec. 19, 2008

Related U.S. Application Data
(60) Provisional application No. 61/009,458, filed on Dec. 28, 2007.

(51) Int. Cl. A61K 51/12; A61K 9/14; A61K 9/10

(52) U.S. Cl. 424/1.29; 424/489; 424/484; 424/486

(57) ABSTRACT

According to an aspect of the invention, injectable particles are provided that include (a) porous polymeric particles that contain at least one type of particle-forming polymer and (b) a pore-filling composition that includes at least one therapeutic agent and at least one pore-filling polymer. The pore-filling composition at least partially fills the pores of the injectable porous polymeric particles. Other aspects of the invention pertain to methods of making such particles.

Still other aspects of the invention pertain to injectable compositions that comprise such particles and to methods of treatment that employ such injectable compositions.

### STATEMENT OF RELATED APPLICATION

This application claims the benefit of U.S. Provisional Patent Application Serial No. 61/009,458, filed Dec. 28, 2007, entitled “Particles For Injection And Processes For Forming The Same,” which is incorporated herein by reference in its entirety.

### FIELD OF THE INVENTION

The invention relates to particles for injection and to processes for forming the same.

### BACKGROUND OF THE INVENTION

Many clinical situations benefit from regulation of the vascular, lymphatic or duct systems by restricting the flow of body fluid or secretions. For example, the technique of embolization involves the therapeutic introduction of particles into the circulation to occlude blood vessels, for example, so as to either arrest or prevent hemorrhaging or to cut off blood flow to a structure or organ. Permanent or temporary occlusion of blood vessels is desirable for managing various diseases, disorders and conditions.

In a typical embolization procedure, local anesthesia is first given over a common artery. The artery is then percutaneously punctured and a catheter is inserted and fluoroscopically guided into the area of interest. An angiogram is then performed by injecting contrast agent through the catheter. An embolic agent is then deposited through the catheter. The embolic agent is chosen, for example, based on the size of the vessel to be occluded, the desired duration of occlusion, and/or the type of abnormality to be treated, among others factors. A follow-up angiogram is usually performed to determine the specificity and completeness of the arterial occlusion.

Various polymer-based microspheres are currently employed to embolize blood vessels. These microspheres are usually introduced to the location of the intended embolization through microcatheters. Current commercially available embolic microspheres are composed of biostable polymers. Materials commonly used commercially for this purpose include polyvinyl alcohol (PVA), acetalized PVA (e.g., Contour SE™ embolic agent, Boston Scientific, Natick, Mass., USA) and crosslinked acrylic hydrogels (e.g., Embospheres®, Biosphere Medical, Rockland, Mass., USA). Similar devices have been used in chemoembolization to increase the residence time of the therapeutic after delivery. In one specific instance, a therapeutic agent (doxorubicin) has been directly added to hydrogel microspheres (prepared from N-acrylamidoacetaldehyde derivatized polyvinyl alcohol copolymerized with 2-acrylamido-2-methylpropane sulfonate) such that the therapeutic agent can be released locally after delivery (e.g., DC Bead™ drug delivery chemoembolization system, Biocompatibles International plc, Famham, Surrey, UK).

It is also known to use polymer-based microspheres as augmentative materials for aesthetic improvement, including improvement of skin contour. Furthermore, polymer-based microspheres have also been used as augmentative materials in the treatment of various diseases, disorders and conditions, including urinary incontinence, vesicourethral reflux, fecal incontinence, intrinsic sphincter deficiency (ISD) and gastro-esophageal reflux disease. For instance, a common method for treating patients with urinary incontinence is via periurethral or transperineal injection of a bulking agent that contains polymer-based microspheres. In this regard, methods of injecting bulking agents commonly require the placement of a needle at a suitable treatment region, for example, periurethrally or transperineally. The bulking agent is injected into a plurality of locations, assisted by visual aids, causing the urethral lining to coapt.

### SUMMARY OF THE INVENTION

According to an aspect of the invention, injectable particles are provided that include (a) porous polymeric particles that contain at least one type of particle-forming polymer and (b) a pore-filling composition that includes at least one therapeutic agent and at least one pore-filling polymer. The pore-filling composition at least partially fills the pores of the injectable porous polymeric particles.

Other aspects of the invention pertain to methods of making such particles.

Still other aspects of the invention pertain to injectable compositions that comprise such particles and to methods of treatment that employ such injectable compositions.

These and various additional aspects, embodiments and advantages of the present invention will become immediately apparent to those of ordinary skill in the art upon review of the Detailed Description and any claims to follow.

### BRIEF DESCRIPTION OF THE DRAWING

FIG. 1 is a schematic illustration of a particle, in accordance with an embodiment of the invention.

### DETAILED DESCRIPTION

In accordance with one aspect of the invention, particulate compositions containing injectable particles are provided in which the injectable particles are porous polymeric particles, which may be formed from one or more types of polymers (also referred to herein as “particle-forming polymers”). The pores of the polymeric particles are at least partially filled with a composition that includes one or more therapeutic agents and one or more types of polymer (also referred to herein as “pore-filling polymers”). The particle-forming polymers may be the same as or different from the pore-filling polymers. The pore-filling polymers may be, for example, previously formed and introduced to the pores or may be formed in the pores via an in situ polymerization process. In addition to residing within the pores of the polymeric particles, the pore-filling polymers may also be present elsewhere, for instance, the pore-filling polymers may be present on the outer surface of the particles and/or may also physically intermingle to a certain degree with the particle-forming polymers. The same is true of the therapeutic agents.

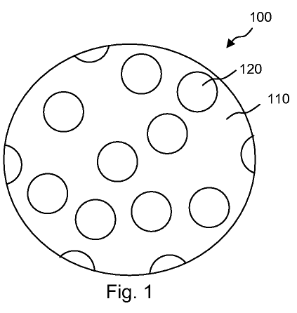

FIG. 1 is a schematic illustration of a particle 100 in accordance with an embodiment of the present invention and shows a porous polymeric particle 110 having pores that are filled with a composition 120 that comprises a therapeutic agent and a pore-filling polymer.

The injectable particles may be used to treat a variety of diseases and conditions in a variety of subjects. Subjects include vertebrate subjects, particularly humans and various warm-blooded animals including pets and livestock. As used herein, “treatment” refers to the prevention of a disease or condition, the reduction or elimination of symptoms associated with a disease or condition, or the substantial or complete elimination of a disease or condition.

The injectable particles of the invention may vary shape. In certain embodiments, they are substantially spherical, for example, having the form of a perfect (to the eye) sphere or the form of a near-perfect sphere such as a prolate spheroid (a slightly elongated sphere) or an oblate spheroid (a slightly flattened sphere). In other embodiments, they are non-spherical, and may be irregular in shape. The injectable particles of the invention can vary in size, with typical longest linear cross-sectional dimensions (e.g., for a sphere, the diameter) ranging, for example, from 40 to 150 to 250 to 500 to 750 to 1000 to 1500 to 2000 to 2500 to 5000 microns (μm).

As used herein a “porous particle” is one that contains pores, which may be observed, for example, by viewing the microspheres using a suitable microscopy technique such as scanning electron microscopy. Pore size may vary widely, ranging from 0.5 micron or less to 1 to 2 microns to 5 microns to 10 microns to 25 microns to 50 microns to 100 microns or more. Pores can come in a wide range of shapes and thus need not be cylindrical. In some embodiments, the particles comprise a porous surface layer disposed over a non-porous core. In other embodiments, pores are present throughout the interior of the particles.

As used herein a “polymeric particle” is one that contains polymers, for example, from 50 wt % or less to 75 wt % to 90 wt % to 95 wt % to 97.5 wt % to 99 wt % or more polymers.

As used herein, “polymers” are molecules that contain multiple copies of one or more types of constitutional units, commonly referred to as monomers. The number of monomers/constitutional units within a given polymer may vary widely, ranging, for example, from 5 to 10 to 25 to 50 to 100 to 1000 to 10,000 or more constitutional units. As used herein, the term “monomers” may refer to the free monomers and those that are incorporated into polymers, with the distinction being clear from the context in which the term is used.

Polymers for use in the present invention can have a variety of architectures, including cyclic, linear and branched architectures. Branched architectures include star-shaped architectures (e.g., architectures in which three or more chains emanate from a single branch point), comb architectures (e.g., architectures having a main chain and a plurality of side chains, such as graft polymers), dendritic architectures (e.g., arborescent and hyperbranched polymers), and networked architectures (e.g., crosslinked polymers), among others.

Polymers containing a single type of monomer are called homopolymers, whereas polymers containing two or more types of monomers are referred to as copolymers. The two or more types of monomers within a given copolymer may be present in any of a variety of distributions including random, statistical, gradient and periodic (e.g., alternating) distributions, among others. One particular type of copolymer is a “block copolymer,” which as used herein is a copolymer that contains two or more polymer chains of different composition, which chains may be selected from homopolymer chains and copolymer chains (e.g., random, statistical, gradient or periodic copolymer chains). As used herein, a polymer “chain” is a linear assembly of monomers and may correspond to an entire polymer or to a portion of a polymer.

As noted above, in the particles of the present invention, the particle-forming polymers may be the same as or different from the pore-filling polymers. As the term is used herein, two polymers are “different” when one polymer comprises a monomer that is not found in the other polymer.

Porous polymeric particles in accordance with the invention may be biostable or biodisintegrable (i.e., particles that disintegrate in vivo due to one or more mechanisms such as dissolution, biodegradation, resorption, etc.).

As used herein, a polymer is “biodegradable” if it undergoes bond cleavage along the polymer backbone in vivo, regardless of the mechanism of bond cleavage (e.g., enzymatic breakdown, hydrolysis, oxidation, etc.).

In some embodiments of the invention, the porous polymeric particles are hydrogel particles. As used herein, a “hydrogel” is a crosslinked hydrophilic polymer (e.g., a polymer network) which swells when placed in water or biological fluids, but remains insoluble due to the presence of crosslinks, which may be, for example, physical, chemical, or both. In some instances, the insolubility of the hydrogel is not permanent, and the particles biodisintegrate in vivo. For instance, a hydrogel particle in accordance with the invention may undergo swelling in water such that its longest linear cross-sectional dimension (e.g., for a sphere, the diameter) increases by 5% or less to 10% to 15% to 20% to 25% or more. A hydrogel particle, as defined herein, also embraces a particle that is capable of absorbing water in an amount such that the water constitutes at least 10% of the total weight of the particle.

Specific polymers for as use as particle-forming polymers or pore-filling polymers in accordance with the invention may be selected, for example, from one or more suitable members of the following, among others: polycarboxylic acid homopolymers and copolymers including polyacrylic acid, polymethacrylic acid, ethylene-methacrylic acid copolymers and ethylene-acrylic acid copolymers, where some of the acid groups can be neutralized with either zinc or sodium ions (commonly known as ionomers); acetal homopolymers and copolymers; acrylate and methacrylate homopolymers and copolymers (e.g., n-butyl methacrylate); cellulosic homopolymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene homopolymers and copolymers; polyimide homopolymers and copolymers such as polyether block imides, polyamidimides, polyesterimides, and polyetherimides; polysulfone homopolymers and copolymers including polyarylsulfones and polyethersulfones; polyamide homopolymers and copolymers including nylon 6,6, nylon 12, polycaprolactams, polyacrylamides and polyether block amides; resins including alkyd resins, phenolic resins, urea resins, melamine resins, epoxy resins, allyl resins and epoxide resins; polycarbonate homopolymers and copolymers; polyacrylonitrile homopolymers and copolymers; polyvinylpyrrolidone homopolymers and copolymers (cross-linked and otherwise); homopolymers and copolymers of vinyl monomers including polyvinyl alcohols, polyvinyl halides such as polyvinyl chlorides, ethylene-vinyl acetate copolymers (EVA), polyvinylidene chlorides, polyvinyl ethers such as polyvinyl methyl ethers, polystyrenes, styrene-maleic anhydride copolymers, vinyl-aromatic-alkylene copolymers, including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene and polystyrene-polyisobutylene-polystyrene (SIBS) block copolymers such as those disclosed in U.S. Pat. No. 6,545,097 to Pinchuk), poly[(styrene-co-p-methylstyrene)-b-isobutylene-b-(styrene-co-p-methylstyrene)] (SMIMS) triblock copolymers described in S. J. Taylor et al., Polymer 45 (2004) 4719-4730; polyphosphonate homopolymers and copolymers; polysulfonate homopolymers and copolymers, for example, sulfonated vinyl aromatic polymers and copolymers, including block copolymers having one or more sulfonated poly(vinyl aromatic) blocks and one or more polyalkene blocks, for example, sulfonated polystyrene-polyolefin-polystyrene triblock copolymers such as the sulfonated SEBS copolymers described in U.S. Pat. No. 5,840,387, and sulfonated versions of SIBS and SMIMS, which polymers may be sulfonated, for example, using the processes described in U.S. Pat. No. 5,840,387 and U.S. Pat. No. 5,468,574, among other sulfonated block copolymers; polyvinyl ketones, polyvinylcarbazoles, and polyvinyl esters such as polyvinyl acetates; polybenzimidazoles; polyalkyl oxide homopolymers and copolymers including polyethylene oxides (PEO); polyesters including polyethylene terephthalates and aliphatic polyesters such as homopolymers and copolymers of lactide (which includes lactic acid as well as d-, l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of poly(lactic acid) and poly(caprolactone) is one specific example); polyether homopolymers and copolymers including polyarylethers such as polyphenylene ethers, polyether ketones, polyether ether ketones; polyphenylene sulfides; polyisocyanates; polyolefin homopolymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated homopolymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone homopolymers and copolymers; thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof; examples of commercially available polyurethane copolymers include Bionate®, Carbothane®, Tecoflex®, Tecothane®, Tecophilic®, Tecoplast®, Pellethane®, Chronothane® and Chronoflex®); p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; polyamine and polyimine homopolymers and copolymers; biopolymers, for example, polypeptides including anionic polypeptides such as polyglutamate and cationic polypeptides such as polylysine, proteins, polysaccharides, and fatty acids (and esters thereof), including fibrin, fibrinogen, collagen, elastin, chitosan, gelatin, starch, glycosaminoglycans such as hyaluronic acid; as well as further copolymers, derivatives (e.g., esters, etc.) and mixtures of the foregoing.

Examples of hydrophilic polymers for as use as particle-forming polymers or pore-filling polymers, not necessarily exclusive of those set forth above, may be selected from suitable members of the following, among many others: homopolymers and copolymers of acrylic acid, methacrylic acid, acrylamides including N-alkylacrylamides, alkylene oxides such as ethylene oxide and propylene oxide, vinyl alcohol, vinyl pyrrolidone, ethylene imine, ethylene amine, acrylonitrile and vinyl sulfonic acid, amino acids such as lysine and glutamic acid and maleic anhydride, hydrophilic polyurethanes, proteins, collagen, cellulosic polymers such as methyl cellulose and carboxymethyl cellulose, dextran, carboxymethyl dextran, modified dextran, alginic acid, pectinic acid, hyaluronic acid, chitin, pullulan, gelatin, gellan, xanthan, starch, carboxymethyl starch, chondroitin sulfate, guar, and further copolymers, derivatives and mixtures of the foregoing. Many of these polymers may be physically crosslinked, chemically crosslinked, or both, to form hydrogels.

Examples of biodegradable polymers for as use as particle-forming polymers or pore-filling polymers, not necessarily exclusive of those set forth above, may be selected from suitable members of the following, among many others: (a) polyester homopolymers and copolymers such as polyglycolide, poly-L-lactide, poly-D-lactide, poly-D,L-lactide, poly(beta-hydroxybutyrate), poly-D-gluconate, poly-L-gluconate, poly-D,L-gluconate, poly(epsilon-caprolactone), poly(delta-valerolactone), poly(p-dioxanone), poly(trimethylene carbonate), poly(lactide-co-glycolide) (PLGA), poly(lactide-co-delta-valerolactone), poly(lactide-co-epsilon-caprolactone), poly(lactide-co-beta-malic acid), poly(lactide-co-trimethylene carbonate), poly(glycolide-co-trimethylene carbonate), poly(beta-hydroxybutyrate-co-beta-hydroxyvalerate), poly[1,3-bis(p-carboxyphenoxy)propane-co-sebacic acid], and poly(sebacic acid-co-fumaric acid), among others, (b) poly(ortho esters) such as those synthesized by copolymerization of various diketene acetals and diols, among others, (c) polyanhydrides such as poly(adipic anhydride), poly(suberic anhydride), poly(sebacic anhydride), poly(dodecanedioic anhydride), poly(maleic anhydride), poly[1,3-bis(p-carboxyphenoxy)methane anhydride], and poly[alpha,omega-bis(p-carboxyphenoxy)alkane anhydrides] such as poly[1,3 -bis(p-carboxyphenoxy)propane anhydride] and poly[1,3-bis(p-carboxyphenoxy)hexane anhydride], among others; and (d) amino-acid-based polymers including tyrosine-based polyarylates (e.g., copolymers of a diphenol and a diacid linked by ester bonds, with diphenols selected, for instance, from ethyl, butyl, hexyl, octyl and bezyl esters of desaminotyrosyl-tyrosine and diacids selected, for instance, from succinic, glutaric, adipic, suberic and sebacic acid), tyrosine-based polycarbonates (e.g., copolymers formed by the condensation polymerization of phosgene and a diphenol selected, for instance, from ethyl, butyl, hexyl, octyl and bezyl esters of desaminotyrosyl-tyrosine), and tyrosine-, leucine- and lysine-based polyester-amides; specific examples of tyrosine-based polymers include includes polymers that are comprised of a combination of desaminotyrosyl tyrosine hexyl ester, desaminotyrosyl tyrosine, and various di-acids, for example, succinic acid and adipic acid, for example, tyrosine-derived ester-amides such as the TyRx 2,2 family of polymers, available from TyRx Pharma, Inc., Monmouth Junction, N.J., USA, among others, as well as further copolymers, derivatives and mixtures of the foregoing.

As indicated above, in accordance with the invention, pores of porous polymeric particles are at least partially filled with a composition comprising one or more therapeutic agents and one or more pore-filling polymers. The therapeutic agents and pore-filling polymers may also be present elsewhere, for instance, present on the exterior surfaces of the particles, intermingled to some degree with the particle-forming polymers (e.g., as a result of diffusion), and so forth.

As seen from the above, the pore-filling polymers may be, for example, hydrophobic, hydrophilic or amphiphilic, they may be charged or uncharged, and they may be biostable or biodisintegrable, among other characteristics.

Similarly, and independently of the pore-filling polymers, the particle-forming polymers may also be, for example, hydrophobic, hydrophilic or amphiphilic, may be charged or uncharged, or may be biostable or biodisintegrable, among other characteristics.

In general, the pore-filling polymers are selected based on their ability to modulate the release of the therapeutic agents from the particles of the invention, for example, increasing, decreasing, or effective preventing the release of the therapeutic agents, relative to what the release characteristics would be in the absence of the pore-filling polymers. Of course, the particle-forming polymers may also influence the release of the therapeutic agents, particularly where the therapeutic agents are intermingled with the particle-forming polymers within the particles.

Among other characteristics, the therapeutic agents may be, for example, hydrophobic, hydrophilic or amphiphilic, and they may be charged or uncharged.

Pore-filling polymers may be selected, for instance, based on their ability to interact with the therapeutic agents in a general or specific fashion, for example, based on non-covalent interactions such as van der Waals forces, hydrophobic interactions and/or electrostatic interactions (e.g., charge-charge interactions, charge-dipole interactions, and dipole-dipole interactions, including hydrogen bonding). Examples of specific non-covalent interactions include π-π stacking, binding based on the formation of multiple hydrogen bonds (e.g., polynucleotide hybridization, etc.), binding based on the formation of complexes and/or coordinative bonds (e.g., metal ion chelation, etc.), binding based on antibody-antigen interactions, also sometimes referred to as antibody-hapten interactions, protein-small molecule interactions (e.g., avidin/streptavidin-biotin binding), protein-protein interactions, and so forth. Specific chemical entities may be covalently attached to the pore-filling polymers for this purpose.

As one example, a pore-filling polymer may be provided with one or more groups (e.g., along the polymer backbone) that electrostatically interact with (e.g., via ion exchange, complexation, coordination, chelation, etc.) a charged therapeutic agent (e.g., a charged radioisotope for radio-embolization therapy). For example, the pore-filling polymer may comprise ligands such as ethylenediamine tetraacetic acid (EDTA) based ligand or acetylacetonate ligands, among others, which are capable of forming coordination compounds (e.g., chelates) with a charged radioactive ion (e.g., yttrium ions).

A benefit of this approach, particularly as it pertains to radioisotopes, is that the various polymers within the particles, including the particle-forming polymers and pore-filling polymers, need not be exposed to high energy radiation associated with the conversion of non-radioactive isotopes (e.g., ⁸⁹Y) to radioactive isotopes (e.g., ⁹⁰Y). Instead, the particles can be loaded with the charged therapeutic agent after it is exposed to the high energy radiation. In this regard, the exposure of many polymers to the levels of radiation needed to convert non-radioactive isotopes to radioactive ones result in significant changes to the polymers (e.g., extensive chain scission and or crosslinking) which would dramatically alter the chemical and/or mechanical properties of the particles.

As noted above, in various embodiments of the invention, the pore-filling polymers may be charged, for example, having cationic groups (e.g., ammonio groups, iminio groups, etc.) (e.g., —NH₃⁺ groups, ═NH₂⁺ groups, ═NH⁺— groups, ═N⁺═ groups, etc.), anionic groups (e.g., carboxylate groups, phosphate groups, sulfonate groups, etc.) (e.g., —COO⁻ groups, —SO₃⁻ groups, —PO₂(OH)⁻ groups etc.), or both. For example, pore-filling polymers may be employed, which have cationic and/or anionic groups along the polymer backbone (e.g., polyamines, polyimines, polycarboxylates, polyphosphates, polysulfonates, etc.). Such charged polymers may be paired with charged therapeutic agents to take advantage of electrostatic interactions. For example, pore-filling polymers having cationic groups may be paired with negatively charged therapeutic agents, or pore-filling polymers having anionic groups may be paired with positively charged therapeutic agents.

A few examples of cationic polymers include salts (e.g., ammonium, lithium, sodium, potassium, etc.) of the following: poly(2-acrylamido-2-methyl-1-propanesulfonic acid), poly(2-acrylamido-2-methyl-1-propanesulfonic acid-co-acrylonitrile), poly(anetholesulfonic acid), poly(4-styrenesulfonic acid), poly(4-styrenesulfonic acid-co-maleic acid), and poly(vinyl sulfonic acid), among others. Examples of anionic polymers include poly(acrylamide-co-diallyldimethylammonium halides), poly(allylamine hydrohalides), poly(diallyldimethylammonium halides), with chloride, bromide and iodide being common halides for use in these polymers, among others. Examples of positively charged therapeutic agents include doxorubicin and campothecin, among others. Examples of negatively charged therapeutic agents include ketorolac and bromopyruvic acid, among others.

For example, in some embodiments an acidic polymer (e.g., one having —COOH groups, —SO₃H groups, —PO(OH)₂ groups, etc.) may be admixed with a basic therapeutic agent and loaded into the particles, or a basic polymer (e.g., one having —NH₂, ═NH or ═N— groups) may be admixed with an acidic therapeutic agent and loaded into the particles.

As another specific example, an amphiphilic pore-filling polymer may be provided, along with a hydrophobic therapeutic agent. In these embodiments, the amphiphilic polymer may form micelles in vivo with a core that corresponds to the hydrophobic therapeutic agent, thereby enhancing release of the therapeutic agent.

To delay release, a hydrophobic pore-filling polymer may be used. For example, a hydrophobic pore-filling polymer may be provided along with a hydrophobic therapeutic agent.

The use of a hydrophilic pore-filling polymer may also delay therapeutic agents release, but to a lesser degree. For example, a hydrophilic pore-filling polymer may be provided along with a hydrophilic therapeutic agent.

The amount of therapeutic agent within the compositions of the present invention will vary widely depending on a number of factors, including the disease, disorder or condition being treated, the potency of the therapeutic agent, and the volume of particulate composition that is ultimately injected into the subject, among other factors. Typical therapeutic agent concentration ranges are, for example, from about 0. 1 to 50 wt % of the particles, among other possibilities.

Examples of therapeutic agents which may be used in the particles of the invention include toxins (e.g., ricin toxin, radioisotopes, or any agents able to kill undesirable, cells such as those making up cancers and other tumors such as uterine fibroids) and agents that arrest growth of undesirable cells.

Some specific examples of therapeutic agents for embolic compositions may be selected from suitable members of the following: radioisotopes (e.g., ⁹⁰Y, ³²P, ¹⁸F, ¹⁴⁰La, ¹⁵³Sm, ¹⁶⁵Dy, ¹⁶⁶Ho, ¹⁶⁹Er, ¹⁶⁹Yb, ¹⁷⁷Lu, ¹⁸⁶Re, ¹⁸⁸Re, ¹⁰³Pd, ¹⁹⁸Au, ¹⁹²Ir, ⁹⁰Sr, ¹¹¹In or ⁶⁷Ga), antineoplastic/antiproliferative/anti-miotic agents including antimetabolites such as folic acid analogs/antagonists (e.g., methotrexate, etc.), purine analogs (e.g., 6-mercaptopurine, thioguanine, cladribine, which is a chlorinated purine nucleoside analog, etc.) and pyrimidine analogs (e.g., cytarabine, fluorouracil, etc.), alkaloids including taxanes (e.g., paclitaxel, docetaxel, etc.), alkylating agents such as alkyl sulfonates, nitrogen mustards (e.g., cyclophosphamide, ifosfamide, etc.), nitrosoureas, ethylenimines and methylmelamines, other aklyating agents (e.g., dacarbazine, etc.), antibiotics and analogs (e.g., daunorubicin, doxorubicin, idarubicin, mitomycin, bleomycins, plicamycin, etc.), platinum complexes (e.g., cisplatin, carboplatin, etc.), antineoplastic enzymes (e.g., asparaginase, etc.), agents affecting microtubule dynamics (e.g., vinblastine, vincristine, colchicine, Epo D, epothilone), caspase activators, proteasome inhibitors, angiogenesis inhibitors (e.g., statins such as endostatin, cerivastatin and angiostatin, squalamine, etc.), rapamycin (sirolimus) and its analogs (e.g., everolimus, tacrolimus, zotarolimus, etc.), etoposides, as well as many others (e.g., hydroxyurea, flavopiridol, procarbizine, mitoxantrone, campothecin, etc.), various pharmaceutically acceptable salts and derivatives (e.g., esters, etc.) of the foregoing, and combinations of the foregoing, among other agents.

Further therapeutic agents include chemical ablation agents (materials whose inclusion in the formulations of the present invention in effective amounts results in necrosis or shrinkage of nearby tissue upon injection) including osmotic-stress-generating agents (e.g., salts, etc.), basic agents (e.g., sodium hydroxide, potassium hydroxide, etc.), acidic agents (e.g., acetic acid, formic acid, etc.), enzymes (e.g., collagenase, hyaluronidase, pronase, papain, etc.), free-radical generating agents (e.g., hydrogen peroxide, potassium peroxide, etc.), other oxidizing agents (e.g., sodium hypochlorite, etc.), tissue fixing agents (e.g., formaldehyde, acetaldehyde, glutaraldehyde, etc.), coagulants (e.g., gengpin, etc.), non-steroidal anti-inflammatory drugs, contraceptives (e.g., desogestrel, ethinyl estradiol, ethynodiol, ethynodiol diacetate, gestodene, lynestrenol, levonorgestrel, mestranol, medroxyprogesterone, norethindrone, norethynodrel, norgestimate, norgestrel, etc.), GnRH agonists (e.g, buserelin, cetorelix, decapeptyl, deslorelin, dioxalan derivatives, eulexin, ganirelix, gonadorelin hydrochloride, goserelin, goserelin acetate, histrelin, histrelin acetate, leuprolide, leuprolide acetate, leuprorelin, lutrelin, nafarelin, meterelin, triptorelin, etc.), antiprogestogens (e.g., mifepristone, etc.), selective progesterone receptor modulators (SPRMs) (e.g., asoprisnil, etc.), various pharmaceutically acceptable salts and derivatives of the foregoing, and combinations of the foregoing, among other agents.

For tissue bulking applications (e.g., urethral bulking, cosmetic bulking, etc.), specific beneficial therapeutic agents include those that promote collagen production, including proinflammatory agents and sclerosing agents such as those listed Pub. No. US 2006/0251697.

Suitable proinflammatory agents can be selected, for example, from suitable endotoxins, cytokines, chemokines, prostaglandins, lipid mediators, and other mitogens. Specific examples of known proinflammatory agents from which suitable proinflammatory agents can be selected include the following: growth factors such as platelet derived growth factor (PDGF), fibroblast growth factor (FGF), transforming growth factor (such as TGF-alpha and TGF-beta), epidermal growth factor (EGF), insulinlike growth factor (IGF), interleukins such as IL-1-(alpha or beta), IL-8, IL-4, IL6, IL-10 and IL-13, tumor necrosis factor (TNF) such as TNF-alpha, interferons such as INF-gamma, macrophage inflammatory protein-2 (MIP-2), leukotrienes such as leukotriene B4 (LTB4), granulocyte macrophage-colony stimulating factor (GM-CSF), cyclooxygenase-1, cyclooxygenase-2, macrophage chemotactic protein (MCP), inducible nitric oxide synthetase, macrophage inflammatory protein, tissue factor, phosphotyrosine phosphates, N-formyl peptides such as formyl-Met-Leu-Phe (fMLP), second mitochondria-derived activator of caspase (sMAC), activated complement fragments (C5a, C3a), phorbol ester (TPA), superoxide, hydrogen peroxide, zymosan, bacterial lipopolysaccharide, imiquimod, various pharmaceutically acceptable salts and derivates of the foregoing, and combinations of the foregoing, among other agents.

Suitable sclerosing agents for the practice of the invention can be selected, for example, from the following (which list is not necessarily exclusive of the pro-inflammatory list set forth above): inorganic materials such as aluminum hydroxide, sodium hydroxide, silver nitrate and sodium chloride, as well as organic compounds, including alcohols such as ethanol, acetic acid, trifluoroacetic acid, formaldehyde, dextrose, polyethylene glycol ethers (e.g., polidocanol, also known as laureth 9, polyethylene glycol (9) monododecyl ether, and hydroxypolyethoxydodecane), tetracycline, oxytetracycline, doxycycline, bleomycin, triamcinolone, minocycline, vincristine, iophendylate, tribenoside, sodium tetradecyl sulfate, sodium morrhuate, diatrizoate meglumine, prolamine diatrizoate, alkyl cyanoacrylates such as N-butyl-2-cyanoactyalte and methyl 2-cyanoacrylate, ethanolamine, ethanolamine oleate, bacterial preparations (e.g., corynebacterium and streptococcal preparations such as picibanil) and mixtures of the same, among others.

Various procedures have associated with them some degree of pain. Thus, in certain embodiments, the injectable particles of the invention contain one or more agents selected from narcotic analgesics, non-narcotic analgesics, local anesthetic agents and other pain management agents.

Examples of narcotic analgesic agents for use in the present invention may be selected from suitable members of the following: codeine, morphine, fentanyl, meperidine, propoxyphene, levorphanol, oxycodone, oxymorphone, hydromorphone, pentazocine, and methadone, among others, as well as combinations and pharmaceutically acceptable salts, esters and other derivatives of the same.

Examples of non-narcotic analgesic agents for use in the present invention may be selected from suitable members of the following: analgesic agents such as acetaminophen, and non-steroidal anti-inflammatory drugs such as aspirin, diflunisal, salsalate, ibuprofen, ketoprofen, naproxen indomethacin, celecoxib, valdecoxib, diclofenac, etodolac, fenoprofen, flurbiprofen, ketorolac, meclofenamate, meloxicam, nabumetone, naproxen, oxaprozin, piroxicam, sulindac, tolmetin, and valdecoxib, among others, as well as combinations and pharmaceutically acceptable salts, esters and other derivatives of the same.

Examples of local anesthetic agents for use in the present invention may be selected from suitable members of the following: benzocaine, cocaine, lidocaine, mepivacaine, and novacaine, among others, as well as combinations and pharmaceutically acceptable salts, esters and other derivatives of the same.

Porous polymeric particles for use in the invention may be formed by any suitable method known in the art. The following discussion pertains to polyols such as polyvinyl alcohol (PVA) for purposes of further illustrating the invention, but the invention is clearly not so-limited.

As noted above, hydrogels are crosslinked hydrophilic polymers (e.g., polymer networks) which swell when placed in water or biological fluids, but remain insoluble due to the presence of crosslinks, which may be, for example, physical, chemical, or both.

Polyols such as PVA can be crosslinked, for example, through the use of chemical crosslinking agents. Some of the common chemical crosslinking agents that have been used for polyol hydrogel preparation include glutaraldehyde, acetaldehyde, formaldehyde, and other monoaldehydes. In the presence of an acid such as sulfuric acid or acetic acid, these crosslinking agents form acetal bridges between the pendant hydroxyl groups found on the polyol chains. For example, acetal formation may proceed to link two alcohol moieties together according to the following scheme:

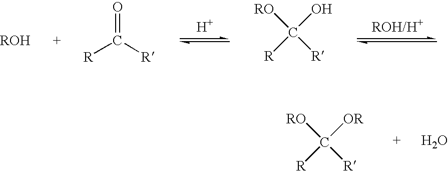

where R and R′ are organic groups. For species with multiple hydroxyl groups, including polyols such as PVA, two hydroxyl groups within the same molecule may react according to the following scheme:

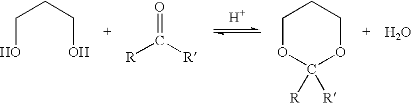

As noted in Pub. No. US 2003/0185895 to Lanphere et al., in certain instances, the reaction of PVA with an aldehyde (formaldehyde) in the presence of an acid is primarily a 1,3 acetalization:

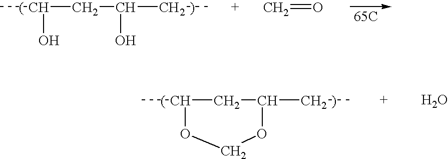

Such intra-chain acetalization reaction can be carried out with relatively low probability of inter-chain crosslinking. Since the reaction proceeds in a random fashion, there will be leftover —OH groups that do not react with adjacent groups.

Other mechanisms of hydrogel preparation involve physical crosslinking due to crystallite formation (e.g., due to freeze-thaw processing) and chemical crosslinking using ionizing radiation such as electron-beam and gamma-ray irradiation. These methods may in some instances be advantageous over techniques that employ chemical cross-linking agents, because they do not leave behind unreacted chemical species.

As a specific example, porous polyol microspheres may be formed as described in Pub. No. US 2003/0185895 to Lanphere et al. Briefly, a solution containing a polyol such as PVA and a gelling precursor such as sodium alginate may be delivered to a viscosity controller, which heats the solution to reduce its viscosity prior to delivery to a drop generator. The drop generator forms and directs drops into a gelling solution containing a gelling agent which interacts with the gelling precursor. For example, in the case where an alginate gelling precursor is employed, an agent containing a divalent metal cation such as calcium chloride may be used as a gelling agent, which stabilizes the drops by gel formation based on ionic crosslinking. The concentration of the gelling agent can control void formation in the particle, thereby controlling the porosity gradient in the particle. Adding non-gelling ions, for example, sodium ions, to the gelling solution can limit the porosity gradient, resulting in a more uniform intermediate porosity throughout the particle. The gel-stabilized drops may then be transferred to a reactor vessel where the polymer in the gel-stabilized drops reacted, thereby forming precursor particles. For example, the reactor vessel may include an agent that chemically reacts with the polyol to cause interchain or intrachain crosslinking. For instance, the vessel may include an aldehyde and an acid, leading to acetalization of the polyol. The precursor particles are then transferred to a gel dissolution chamber, where the gel is dissolved. For example, ionically crosslinked alginate may be removed by ion exchange with a solution of sodium hexa-metaphosphate. Alginate may also be removed by radiation degradation. Porosity is generated due to the presence (and ultimate removal) of the alginate. The particles may then be filtered to remove any residual debris and to sort the particles into desired size ranges.

Using the above and other techniques, porous particles may be formed having a variety of pore sizes and porosities. Moreover, porous acetalized PVA particles are commercially available (e.g., Contour® embolic agent, Boston Scientific, Natick, Mass., USA). Once porous polymeric particles of suitable size and porosity are obtained, in accordance with an aspect of the invention, the pores of the particles are at least partially filled with a composition comprising one or more therapeutic agents and one or more pore-filling polymers.

In one method, one or more monomers is/are provided within the pores of the porous polymeric particles and polymerized in situ. This may be either preceded or succeeded by introduction of one or more therapeutic agents.

In another method, porous polymeric particles are exposed to a solution containing one or more therapeutic agents and one or more pore-filling polymers.

In another method, porous polymeric particles are exposed to a first solution containing one or more pore-filling polymers, followed by a second solution containing one or more therapeutic agents, or vice versa. The porous polymeric particles may be contacted with the solutions in wet or dry form.

Depending on the nature of the porous polymeric particles, the pore-filling polymers and the therapeutic agents, the solvent systems used to create the above solutions may be based on (a) water, (b) one or more organic solvents, or (c) water and one or more organic solvents. Ideally, the one or more pore-filling polymers should be soluble in the selected solvent system. Moreover, the one or more therapeutic agents should be soluble (or at least dispersible) in the selected solvent system. Furthermore, the selected solvent system should not destroy the integrity of the porous polymeric particles. In some embodiments, a solvent system is selected that swells the particles to some degree.

In those specific embodiments where the porous polymeric particles are hydrogels, the solvent system may be one based on water, one or more polar organic solvents (e.g., ethanol, methanol, propanol, or isopropanol), or water plus one or more polar organic solvents. Polar organic solvents may be used, for example, in conjunction with the loading of hydrophobic pore-filling polymers and/or hydrophobic therapeutic agents.

In some embodiments of the invention, the pore filling polymer may be covalently bound to the porous polymeric particles. The pore filling polymer may be introduced after the particle formation process, for example, by bringing the pore filling polymer to be covalently bonded into contact with the particles. This may be achieved, for example, by exposing the porous polymeric particles to a solution of the pore filling polymer. Subsequently, the pore filling polymer is covalently bonded to the polymers within the particles. For example, the pore filling polymer and the porous polymeric particles may be covalently bound by exposure to a suitable type of radiation (e.g., electron beam radiation, gamma radiation, UV radiation, etc.). As one specific example, gamma radiation or an electron beam can be used to covalently bond polymeric styrene sulfonic acid, acrylic acid, vinyl amine, vinyl pyrrolidone, or dimethylaminoethylacrylate to formalized or unformalized PVA.

The particles of the invention may be stored and transported in dry form. The dry composition may also optionally contain additional agents, for example, one or more of the following among others: (a) tonicity adjusting agents including sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), (b) suspension agents including various surfactants, wetting agents, and polymers (e.g., albumen, PEO, polyvinyl alcohol, block copolymers, etc.), (c) imaging contrast agents (e.g., Omnipaque™, Visipaque™, etc.), and (d) pH adjusting agents including various buffer solutes. The dry composition may shipped, for example, in a syringe, catheter, vial, ampoule, or other container, and it may be mixed with an appropriate liquid carrier (e.g. sterile water for injection, physiological saline, phosphate buffer, a solution containing an imaging contrast agent, etc.) prior to administration. In this way the concentration of the composition to be injected may be varied at will, depending on the specific application at hand, as desired by the health care practitioner in charge of the procedure. One or more containers of liquid carrier may also be supplied and shipped, along with the dry particles, in the form of a kit.

The particles of the invention may also be stored and transported in wet form. For example, the injectable particles may be stored in a suspension that contains water in addition to the particles themselves, as well as other optional agents such as one or more of the tonicity adjusting agents, suspension agents, contrast media, and pH adjusting agents listed above, among others. The suspension may be stored, for example, in a syringe, catheter, vial, ampoule, or other container. The suspension may also be mixed with a suitable liquid carrier (e.g. sterile water for injection, physiological saline, phosphate buffer, a solution containing contrast agent, etc.) prior to administration, allowing the concentration of administered particles (as well as other optional agents) in the suspension to be reduced prior to injection, if so desired by the health care practitioner in charge of the procedure. One or more containers of liquid carrier may also be supplied to form a kit.

The amount of injectable particles within a suspension to be injected may be determined by those of ordinary skill in the art. The amount of particles may be limited by the fact that when the amount of particles in the composition is too low, too much liquid may be injected, possibly allowing particles to stray far from the site of injection, which may result in undesired embolization or bulking of vital organs and tissues. When the amount of particles is too great, the delivery device (e.g., catheter, syringe, etc.) may become clogged.

An effective amount of the particle compositions of the invention is, for example, (a) an amount sufficient to produce an occlusion or emboli at a desired site in the body, (b) an amount sufficient to achieve the degree of bulking desired (e.g., an amount sufficient to improve urinary incontinence, vesicourethral reflux, fecal incontinence, ISD or gastro-esophageal reflux, or an amount sufficient for aesthetic improvement), or (c) an amount sufficient to locally treat a disease, disorder or condition. Effective doses may also be extrapolated from dose-response curves derived from animal model test systems, among other techniques.

In certain embodiments, the density of the aqueous phase that suspends the particles is close to that of the particles themselves, thereby promoting an even suspension. The density of the aqueous phase may be increased, for example, by increasing the amount of solutes that are dissolved in the aqueous phase, and vice versa.

As noted above, permanent or temporary occlusion of blood vessels is useful for managing various diseases, disorders and conditions. For example, fibroids, also known as leiomyoma, leiomyomata or fibromyoma, are the most common benign tumors of the uterus. These non-cancerous growths are present in significant fraction of women over the age of 35. In most cases, multiple fibroids are present, often up to 50 or more. Fibroids can grow, for example, within the uterine wall (“intramural” type), on the outside of he uterus (“subserosal” type), inside the uterine cavity (“submucosal” type), between the layers of broad ligament supporting the uterus (“interligamentous” type), attached to another organ (“parasitic” type), or on a mushroom-like stalk (“pedunculated” type). Fibroids may range widely in size, for example, from a few millimeters to 40 centimeters. In some women, fibroids can become enlarged and cause excessive bleeding and pain. While fibroids have been treated in the past by surgical removal of the fibroids (myomectomy) or by removal of the uterus (hysterectomy), recent advances in uterine embolization now offer a nonsurgical treatment. Thus, injectable compositions in accordance with the present invention can be used to treat uterine fibroids.

Methods for treatment of fibroids by embolization are well known to those skilled in the art (see, e.g., Pub. No. US 2003/0206864 to Mangin and the references cited therein). Uterine embolization is aimed at starving fibroids of nutrients. Numerous branches of the uterine artery may supply uterine fibroids. In the treatment of fibroids, embolization of the entire uterine arterial distribution network is often preferred. This is because it is difficult to selectively catheterize individual vessels supplying only fibroids, the major reason being that there are too many branches for catheterization and embolization to be performed in an efficient and timely manner. Also, it is difficult to tell whether any one vessel supplies fibroids rather than normal myometrium. In many women, the fibroids of the uterus are diffuse, and embolization of the entire uterine arterial distribution affords a global treatment for every fibroid in the uterus.

In a typical procedure, a catheter is inserted near the uterine artery by the physician (e.g., with the assistance of a guide wire). Once the catheter is in place, the guide wire is removed and contrast agent is injected into the uterine artery. The patient is then subjected to fluoroscopy or X-rays. In order to create an occlusion, an embolic agent is introduced into the uterine artery via catheter. The embolic agent is carried by the blood flow in the uterine artery to the vessels that supply the fibroid. The particles flow into these vessels and clog them, thus disrupting the blood supply to the fibroid. In order for the physician to view and follow the occlusion process, contrast agent may be injected subsequent to infusion of the embolic agent. Treatment is enhanced in the present invention by the therapeutic agent (e.g., antineoplastic/antiproliferative/ anti-miotic agent, toxin, ablation agent, etc.) that is present in the particles.

Controlled, selective obliteration of the blood supply to tumors is also used in treating solid tumors such as renal carcinoma, bone tumor and liver cancer, among various others. The idea behind this treatment is that preferential blood flow toward a tumor will carry the embolization agent to the tumor thereby blocking the flow of blood which supplies nutrients to the tumor, thus, causing it to shrink. Embolization may be conducted as an enhancement to chemotherapy or radiation therapy. Treatment is enhanced in the present invention by the therapeutic agent (e.g., antineoplastic/antiproliferative/anti-miotic agent, toxin, ablation agent, etc.) that is present in the particles.

Particle compositions in accordance with the invention may also be used to treat various other diseases, conditions and disorders, including treatment of the following: arteriovenous fistulas and malformations including, for example, aneurysms such as neurovascular and aortic aneurysms, pulmonary artery pseudoaneurysms, intracerebral arteriovenous fistula, cavernous sinus dural arteriovenous fistula and arterioportal fistula, chronic venous insufficiency, varicocele, pelvic congestion syndrome, gastrointestinal bleeding, renal bleeding, urinary bleeding, varicose bleeding, uterine hemorrhage, and severe bleeding from the nose (epistaxis), as well as preoperative embolization (to reduce the amount of bleeding during a surgical procedure) and occlusion of saphenous vein side branches in a saphenous bypass graft procedure, among other uses. As elsewhere herein, treatment is enhanced in the present invention by the therapeutic agent that is present in the particles.

Particle compositions in accordance with the invention may also be used in tissue bulking applications, for example, as augmentative materials in the treatment of urinary incontinence, vesicourethral reflux, fecal incontinence, intrinsic sphincter deficiency (ISD) or gastro-esophageal reflux disease, or as augmentative materials for aesthetic improvement. For instance, a common method for treating patients with urinary incontinence is via periurethral or transperineal injection of a bulking material. In this regard, methods of injecting bulking agents commonly require the placement of a needle at a treatment region, for example, periurethrally or transperineally. The bulking agent is injected into a plurality of locations, assisted by visual aids, causing the urethral lining to coapt. In some cases, additional applications of bulking agent may be required. Treatment is enhanced in the present invention by the therapeutic agent (e.g., proinflammatory agents, sclerosing agents, etc.) that is present in the particles.

The present invention encompasses various ways of administering the particulate compositions of the invention to effect embolization, bulking or other procedure benefiting from therapeutic agent release. One skilled in the art can determine the most desirable way of administering the particles depending on the type of treatment and the condition of the patient, among other factors. Methods of administration include, for example, percutaneous techniques as well as other effective routes of administration. For example, the particulate compositions of the invention may be delivered through a syringe or through a catheter, for instance, a FasTracker® microcatheter (Boston Scientific, Natick, Mass., USA), which can be advanced over a guidewire, a steerable microcatheter, or a flow-directed microcatheter (MAGIC, Balt, Montomorency, France).

Various aspects of the invention of the invention relating to the above are enumerated in the following paragraphs:

Aspect 1. Injectable particles comprising (a) porous polymeric particles that comprise a particle-forming polymer and (b) a composition that comprises a therapeutic agent and a pore-filling polymer, said composition at least partially filling the pores of the injectable porous polymeric particles, wherein the particle-forming polymer may the same as or different from the pore-filling polymer.

Aspect 2. The injectable particles of Aspect 1, wherein 95 vol % of the particles have a longest linear cross-sectional dimension between 40 μm and 5000 μm.

Aspect 3. The injectable particles of Aspect 1, wherein the particles are spherical.

Aspect 4. The injectable particles of Aspect 3, wherein 95 vol % of the particles have a longest linear cross-sectional dimension between 40 μm and 5000 μm

Aspect 5. The injectable particles of Aspect 1, wherein the particles are non-spherical.

Aspect 6. The injectable particles of Aspect 5, wherein 95 vol % of the particles have a longest linear cross-sectional dimension between 40 μm and 5000 μm.

Aspect 7. The injectable particles of Aspect 1, wherein the particles comprise pores ranging from 0.5 to 100 μm in width.

Aspect 8. The injectable particles of Aspect 1, wherein the porous polymeric particles are biostable.

Aspect 9. The injectable particles of Aspect 1, wherein the porous polymeric particles are biodisintegrable.

Aspect 10. The injectable particles of Aspect 1, wherein the porous polymeric particles are hydrogel particles.

Aspect 11. The injectable particles of Aspect 10, wherein the porous polymeric particles comprise crosslinked polyvinyl alcohol as a particle-forming polymer.

Aspect 12. The injectable particles of Aspect 1, wherein the therapeutic agent is selected from toxins, antineoplastic agents, ablation agents, proinflammatory agents and sclerosing agents.

Aspect 13. The injectable particles of Aspect 1, wherein the pore-filling polymer is biostable.

Aspect 14. The injectable particles of Aspect 1, wherein the pore-filling polymer is biodisintegrable.

Aspect 15. The injectable particles of Aspect 1, wherein the pore-filling polymer is hydrophobic and the therapeutic agent is hydrophobic.

Aspect 16. The injectable particles of Aspect 1, wherein the pore-filling polymer is an amphiphilic and the therapeutic agent is hydrophobic.

Aspect 17. The injectable particles of Aspect 1, wherein the pore-filling polymer is hydrophilic and the therapeutic agent is hydrophilic.

Aspect 18. The injectable particles of Aspect 1, wherein the therapeutic agent is charged and the pore-filling polymer non-covalently binds to the therapeutic agent by electrostatic interactions.

Aspect 19. The injectable particles of Aspect 18, wherein the therapeutic agent is a charged radioisotope and the pore-filling polymer comprises ligands that form a coordination complex with the charged radioisotope.

Aspect 20. The injectable particles of Aspect 18, wherein the therapeutic agent is a charged organic compound and the pore-filling polymer comprises a net charge that is opposite to that of the charged organic compound.

Aspect 21. The injectable particles of Aspect 17, wherein the pore-filling polymer comprises pendant groups selected from —COO⁻ groups, —SO₃⁻ groups, —PO₂(OH)⁻ groups, —NH₃⁺ groups, ═NH₂⁺ groups, ═NH⁺— groups, ═N⁺═ groups, and combinations thereof.

Aspect 22. An injectable medical composition comprising the particles of Aspect 1.

Aspect 23. The injectable medical composition of Aspect 22, comprising a tonicity adjusting agent.

Aspect 24. The injectable medical composition of Aspect 23, wherein the tonicity adjusting agent is selected from sugars, polyhydric alcohols, inorganic salts and combinations thereof.

Aspect 25. The injectable medical composition of Aspect 22, wherein the injectable medical composition is disposed within a glass container or a preloaded syringe.

Aspect 26. A method of forming the injectable particles of Aspect 1, comprising exposing porous polymeric particles to a solution comprising the therapeutic agent and the pore-filling polymer.

Aspect 27. The method of Aspect 26, wherein wet or dry porous polymeric particles are exposed to the solution.

Aspect 28. A method of forming the injectable particles of Aspect 1, comprising (a) exposing porous polymeric particles to a solution comprising the pore-filling polymer and (b) exposing the resulting particles to a solution comprising the therapeutic agent.

### EXAMPLES

### Reagents:

CSE Contour Spherical Embolization microspheres (100-300 μm), Boston Scientific, Natick, Mass., USA.

Poly (sodium-4-styrene sulfonate), 30 weight % solution in water, Part #561967, Sigma Aldrich, Milwaukee, Wis., USA.

Poly (vinylsulfonic acid, sodium salt), 25 weight % solution in water, Part #278424, Sigma Aldrich, Milwaukee, Wis., USA.

Poly (acrylic acid, sodium salt), 45 weight % solution in water, MW-8000, Sigma Aldrich, Milwaukee, Wis., USA.

Poly (acrylic acid, sodium salt), 45 weight % solution in water, MW-12000, Sigma Aldrich Milwaukee, Wis., USA.

Poly (acrylic acid, sodium salt), 35 weight % solution in water, MW-15000, Sigma Aldrich Milwaukee, Wis., USA.

Adriamycin®, Doxorubicin Hydrochloride (HCl), 50 mg lyophilized powder, Bedford Labs, Bedford, Ohio, USA.

### Instruments:

Synergy™ 2 Microplate Reader, BIOTEK Instruments, Winooski, Vt., USA.

### Example 1

Polymers were grafted to microspheres following the general procedure described below. Specific amounts of reagent and specific reaction conditions are listed in Table I for individual examples. Polymer loading solution of appropriate weight percent concentration (Table I) was prepared by dissolving weighed polymer into deionized (DI) water.

Clear vial(s) containing either 1 ml of wet CSE microspheres or 100 mg dry (lyophilized) microspheres were prepared. Saline was removed from wet CSE microspheres using a syringe with a small gauge needle. About 5 ml of polymer loading solution was added to the vial(s) containing drained-wet or dry microspheres, and the mixture was kept in an incubator-shaker (MAX^{Q} 4000—A Class, Barnstead Lab Line, Dubuque, Iowa, USA) under the conditions specified in Table I. The vial(s) containing the polymer-microsphere mixture were nitrogen purged to remove excess oxygen and then treated with E-beam radiation at specified dose(s). The e-beamed mixture of polymer and microspheres were washed repeatedly with DI water. The vials were refilled with 1 mL of washed microspheres and 5 ml of saline and then re-treated with E-beam radiation. Selected samples were then washed with water and freeze dried for analysis by sulfur combustion (Galbraith Laboratories Inc. Knoxville, Tenn.).

TABLE 1

| | Microsphere | | | Percent | | | E-beam radiation | | | |
|---|---|---|---|---|---|---|---|---|---|---|
| | weight/ | | | Polymer | Incubation | | | Number of | | Analysis |
| | volume | | | in DI | conditions | | | treatments | Washing | % Sulfur |
| Example | Wet | Dry | Polymer Type | water | Time | Temperature | Dose | (X) | cycles | Detected |
| Control | 1 ml | 100 mg | n/a | n/a | n/a | n/a | n/a | n/a | n/a | <0.3% |
| (CSE | | | | | | | | | | |
| microspheres) | | | | | | | | | | |
| 1a | 1 ml | 100 mg | Poly (sodium- | 1% | 4-24 hrs | 37° C. | 25 | 1X | 10 | 0.2% |
| 2a | | | 4-styrene | 5% | | | KGY | | | 0.3% |
| 3a | | | sulfonate) | 10% | | | | | | 0.4% |
| | | | or | | | | | | | |
| | | | Poly (vinyl | | | | | | | |
| | | | sulfonic acid, | | | | | | | |
| | | | sodium salt) | | | | | | | |
| 4a | 1 ml | 100 mg | Poly (acrylic | 0.5% to | 4-24 hrs | 37° C. | 25 | 1X | 10 | N/A |
| | | | acid, sodium | 5% | | | KGY | | | |
| | | | salt) | | | | | | | |
| | | | MW - 8000 to | | | | | | | |
| | | | 15000 | | | | | | | |

### Example 2

Doxorubicin HCl was loaded onto microspheres following the general procedure below. Specific amounts of reagent and specific reaction conditions are listed in Table 2 for individual examples.

Each 50 mg doxorubicin HCl vial (Adriamycin®) was reconstituted with an appropriate volume of saline to get the required concentration (2 mg/ml for 8 mg or 4 mg/ml for 16 mg of drug loading solution) and mixed well until a clear solution was obtained. Saline was removed from vial(s) containing 1 ml of polymer grafted microspheres made in accordance with Example 1 using a syringe with a small gauge needle. Using a syringe and needle 4 ml of reconstituted Doxorubicin HCl solution was added to the drained vial(s) of microspheres. The microsphere/ doxorubicin HCl solution was agitated gently by hand to encourage mixing and then allowed to stand for 30 minutes with gentle agitation every 5-7 minutes. Excess doxorubicin solution was removed using a syringe and needle or a vacuum filter to collect the loading solution. The collected/filtered loading solution was analyzed for doxorubicin content by fluorescence spectroscopy using a Synergy™ 2 Microplate Reader, BIOTEK Instruments, Winooski, Vt., USA at an excitation/emission of 485/590 respectively.

TABLE 2

| | | | | | | | Doxorubicin | |
|---|---|---|---|---|---|---|---|---|
| | | | Loading | Doxorubicin | | | HCl uptake | |
| | | Microsphere | solution | HCl (amount | | | (mg/ml | |
| Example | Sample | volume | volume | added, mg) | Time | Temp. | microsphere) | |
| Control-1 | Control | 1 ml | 4 ml | 8 | 30 minutes | 25° C. | 2 | mg |
| Control-2 | Control | 1 ml | 4 ml | 16 | 30 minutes | 25° C. | 7 | mg |
| 1b-1 | 1a | 1 ml | 4 ml | 8 | 30 minutes | 25° C. | 2 | mg |
| 1b-2 | 1a | 1 ml | 4 ml | 16 | 30 minutes | 25° C. | 6 | mg |
| 2b-1 | 2a | 1 ml | 4 ml | 8 | 30 minutes | 25° C. | 3 | mg |
| 2b-2 | 2a | 1 ml | 4 ml | 16 | 30 minutes | 25° C. | 6 | mg |
| 3b-1 | 3a | 1 ml | 4 ml | 8 | 30 minutes | 25° C. | 6 | mg |
| 3b-2 | 3a | 1 ml | 4 ml | 16 | 30 minutes | 25° C. | 7 | mg |
| 4b | 4a | 1 ml | 4 ml | 16 | 30 minutes | 25° C. | 6-8 | mg |

### Example 3

Doxorubicin HCl was released from microspheres following the general, in vitro procedure below. Specific amounts of reagent and specific reaction conditions are listed in Table 3 for individual examples.

Drug loaded microspheres of Example 2 equivalent to 1 ml (filtered or drained) were collected into centrifuge tube(s) and 10 ml of freshly prepared phosphate buffer solution with 1% Tween 20, pH-7.4 (PBS-Tween 20 media) was added into the tube(s). The tubes were kept in incubator-shaker at 37° C. and 150 RPM until further sampling. Using a syringe and needle 2 ml sample(s) of solution from the tube(s) were taken at pre-determined time intervals. A 2 ml aliquot of fresh PBS-Tween 20 media was added to the tube(s) after each sampling. The samples were analyzed for doxorubicin content by fluorescence spectroscopy at an excitation/emission of 485/590 respectively.

TABLE 3

| | | | | Dox Release | Dox Release |
|---|---|---|---|---|---|
| | | Dox Release | Dox Release | (mg) at 7 | (mg) at 14 |
| Example | Sample | (mg) at 1 hour | (mg) at 1 Day | Days | Days |
| Control-1R | Control-1 | 1.37 | 1.60 | 1.68 | 1.70 |
| Control-2R | Control-2 | 1.87 | 2.15 | 2.24 | 2.26 |
| 1c-1 | 1b-1 | 1.42 | 1.67 | 1.76 | 1.76 |
| 1c-2 | 1b-2 | 1.92 | 2.20 | 2.30 | 2.32 |
| 2c-1 | 2b-1 | 1.50 | 1.78 | 1.89 | 1.90 |
| 3c-1 | 3b-1 | 1.38 | 1.65 | 1.75 | 1.76 |
| 3c-2 | 3b-2 | 2.00 | 2.30 | 2.39 | 2.42 |
| 4c | 4b | 1.56 | 1.87 | 1.88 | 1.88 |

### Example 4

Contour SE™ microspheres, 500-700 urn (Boston Scientific, Natick, Mass., USA) are lyophilized to provide a dried porous microsphere composition. The dried porous microspheres are re-hydrated in an aqueous solution containing 25% by weight polyacrylic acid. After 24 hours the microspheres are removed from the solution, washed briefly with deionized water and then lyophilized to yield dry, composite microspheres. The composite microspheres are then loaded by exposure to a solution containing a therapeutic agent of choice.

### Example 5

Contour SE™ microspheres, 500-700 urn (Boston Scientific, Natick, Mass., USA) are lyophilized to provide a dried porous microsphere composition. The dried porous microspheres are dispersed in an acetone solution containing 25% by weight poly(4-vinylpyridine). After 24 hours the microspheres are removed from the solution, washed briefly with acetone and then dried to yield dry, composite microspheres. The composite microspheres are then loaded by exposure to a solution containing a therapeutic agent of choice.

Although various aspects and embodiments are specifically illustrated and described herein, it will be appreciated that modifications and variations of the present invention are covered by the above teachings and are within the purview of any appended claims without departing from the spirit and intended scope of the invention.

1. Injectable particles comprising (a) porous polymeric particles that comprise a particle-forming polymer and (b) a composition that comprises a therapeutic agent and a pore-filling polymer, said composition at least partially filling the pores of the injectable porous polymeric particles, wherein the particle-forming polymer may the same as or different from the pore-filling polymer.

2. The injectable particles of claim 1, wherein 95 vol % of said particles have a longest linear cross-sectional dimension between 40 μm and 5000 μm.

3. The injectable particles of claim 1, wherein said particles are spherical.

4. The injectable particles of claim 3, wherein 95 vol % of said particles have a longest linear cross-sectional dimension between 40 μm and 5000 μm.

5. The injectable particles of claim 1, wherein said particles are non-spherical.

6. The injectable particles of claim 5, wherein 95 vol % of said particles have a longest linear cross-sectional dimension between 40 μm and 5000 μm.

7. The injectable particles of claim 1, wherein said particles comprise pores ranging from 0.5 to 100 μm in width.

8. The injectable particles of claim 1, wherein said porous polymeric particles are biostable.

9. The injectable particles of claim 1, wherein said porous polymeric particles are biodisintegrable.

10. The injectable particles of claim 1, wherein said porous polymeric particles are hydrogel particles.

11. The injectable particles of claim 10, wherein said porous polymeric particles comprise crosslinked polyvinyl alcohol as a particle-forming polymer.

12. The injectable particles of claim 1, wherein said therapeutic agent is selected from toxins, antineoplastic agents, ablation agents, proinflammatory agents and sclerosing agents.

13. The injectable particles of claim 1, wherein said pore-filling polymer is biostable.

14. The injectable particles of claim 1, wherein said pore-filling polymer is biodisintegrable.

15. The injectable particles of claim 1, wherein said pore-filling polymer is hydrophobic and the therapeutic agent is hydrophobic.

16. The injectable particles of claim 1, wherein said pore-filling polymer is an amphiphilic and the therapeutic agent is hydrophobic.

17. The injectable particles of claim 1, wherein said pore-filling polymer is hydrophilic and the therapeutic agent is hydrophilic.

18. The injectable particles of claim 1, wherein said therapeutic agent is charged and the pore-filling polymer non-covalently binds to the therapeutic agent by electrostatic interactions.

19. The injectable particles of claim 18, wherein said therapeutic agent is a charged radioisotope and the pore-filling polymer comprises ligands that form a coordination complex with the charged radioisotope.

20. The injectable particles of claim 18, wherein said therapeutic agent is a charged organic compound and the pore-filling polymer comprises a net charge that is opposite to that of the charged organic compound.

21. The injectable particles of claim 17, wherein said pore-filling polymer comprises pendant groups selected from —COO⁻ groups, —SO₃⁻ groups, —PO₂(OH)⁻ groups, —NH₃ groups, ═NH₂⁺ groups, ═NH⁺— groups, ═N⁻═ groups, and combinations thereof.

22. An injectable medical composition comprising the particles of claim 1.

23. The injectable medical composition of claim 22, comprising a tonicity adjusting agent.

24. The injectable medical composition of claim 23, wherein said tonicity adjusting agent is selected from sugars, polyhydric alcohols, inorganic salts and combinations thereof.

25. The injectable medical composition of claim 22, wherein said injectable medical composition is disposed within a glass container or a preloaded syringe.

26. A method of forming the injectable particles of claim 1, comprising exposing porous polymeric particles to a solution comprising said therapeutic agent and said pore-filling polymer.

27. The method of claim 26, wherein wet or dry porous polymeric particles are exposed to said solution.

28. A method of forming the injectable particles of claim 1, comprising (a) exposing porous polymeric particles to a solution comprising said pore-filling polymer and (b) exposing the resulting particles to a solution comprising said therapeutic agent.
